(19) United States
(12) Patent Application Publication
(10) Pub. No.: US 2023/0037614 A1
(43) Pub. Date: Feb. 9, 2023

(54) METAL OXIDE PARTICLES COATED WITH A RARE-EARTH OXIDE AND PROCESS FOR PREPARING SAME BY FLAME SPRAY PYROLYSIS

(71) Applicant: L'OREAL, Paris (FR)

(72) Inventors: Valérie JEANNE-ROSE, AULNAY-SOUS-BOIS (FR); Henri SAMAIN, CHEVILLY-LARUE (FR); Yiannis DELIGIANNAKIS, IOANNINA (GR); Maria LOULOUDI, IOANNINA (GR)

(73) Assignee: L'OREAL, Paris (FR)

(21) Appl. No.: 17/789,501

(22) PCT Filed: Dec. 24, 2020
(86) PCT No.: PCT/EP2020/087876

§ 371 Date: Jun. 27, 2022

(30) Foreign Application Priority Data
Dec. 27, 2019 (FR) 1915681

(51) Int. Cl. A61K 8/02; A61Q 15/00; A61Q 17/04; A61K 8/29; A61K 8/19

(52) U.S. Cl. CPC A61K 8/0241; A61Q 15/00; A61Q 17/04; A61K 8/29; A61K 8/19; A61K 2800/621; A61K 2800/651

(57) ABSTRACT

The present invention relates to coated metal oxide particles, to a process for preparing such coated particles by means of flame spray pyrolysis technology, to metal oxide particles derived from such a process, to the compositions comprising such particles and also to the uses thereof.

The present invention relates to coated metal oxide particles, to a process for preparing such coated particles by means of flame spray pyrolysis technology, to metal oxide particles derived from such a process, to the compositions comprising such particles and also to the uses thereof.

Metal oxides are used in many applications (cosmetics, paints, stains, electronics, rubber, etc.), notably for their optical properties. In particular, use is made of their light absorption and/or light scattering properties in order to protect surfaces from UV radiation and/or in order to convert ambient light into electricity.

However, metal oxides have the drawback of being particularly unstable over time. By way of example, zinc oxide may degrade to zinc hydroxide, or even to Zn²⁺ ion, in the presence of water originating from the composition comprising it or from atmospheric moisture. Such a degradation leads to a partial or even total solubilization of the zinc oxide in water and has the effect of greatly reducing, or even removing, the desired properties of the zinc oxide.

This instability is particularly problematic when the metal oxides are used in photoprotective cosmetic compositions. Indeed, the ultraviolet radiation protection decreases as the metal oxides degrade.

It has been envisaged to coat the metal oxides with silica, notably using sol-gel processes, or else to graft fluoro compounds onto the metal oxides. However, these solutions are not entirely satisfactory. The metal oxides coated with silica via a sol-gel process generally have worse optical properties than an uncoated particle. As for the grafting technique, the use of fluoro compounds may be harmful to the environment and dangerous for the user.

It is also known to use a flame spray pyrolysis method (FSP method) to prepare metal oxide particles.

Flame spray pyrolysis or FSP is a well-known method these days, which was essentially developed for the synthesis of ultrafine powders of single or mixed oxides of various metals (e.g. SiO₂, Al₂O₃, B₂O₃, ZrO₂, GeO₂, WO₃, Nb₂O₅, SnO₂, MgO, ZnO), with controlled morphologies, and/or the deposition thereof on various substrates, by starting from a wide variety of metal precursors, generally in the form of organic or inorganic, preferably inflammable, sprayable liquids; the liquids sprayed into the flame, by being burnt, notably emit nanoparticles of metal oxides which are sprayed by the flame itself onto these various substrates. The principle of this method has been recalled for example in the recent (2011) publication by Johnson Matthey entitled “Flame Spray Pyrolysis: a Unique Facility for the Production of Nanopowders”, Platinum Metals Rev., 2011, 55, (2), 149-151. Numerous variants of FSP processes and reactors have also been described, by way of example, in the patents or patent applications: U.S. Pat. Nos. 5,958,361, 2,268,337, WO 01/36332 or U.S. Pat. No. 6,887,566, WO 2004/005184 or U.S. Pat. No. 7,211,236, WO 2004/056927, WO 2005/103900, WO 2007/028267 or U.S. Pat. No. 8,182,573, WO 2008/049954 or U.S. Pat. No. 8,231,369, WO 2008/019905, US 2009/0123357, US 2009/0126604, US 2010/0055340, WO 2011/020204.

However, this method, applied to the preparation of metal oxide can still be perfected, notably in order to improve the stability of the metal oxide particles over time, and more particularly, its water resistance.

Furthermore, the scientific article by Han Gao et al describes particles involving placing a layer of CeO₂ on TiO₂ particles in order to reduce the formation of the radicals resulting from the interaction between TiO₂, light and the external environment. However, this type of particle is coated with an extremely thin layer of cerium oxide which does not completely cover the TiO₂ core. (Ind. Eng. Chem. Res. 2014, 53(1), 189-197). These particles are not suitable since the cerium oxide layer does not cover the TiO₂ core, does not make it possible to prevent the titanium oxide from coming into contact with the external environment and the release of titanium atoms being found in the particle core in particular. Another scientific article concerns particles that combine an iron oxide core and a coating formed of cerium oxide and acrylate polymer as theranostic material for inflammatory diseases (Y. Wu et al. Journal of Materials Chemistry B. 2018, 6, pp 4937-4951). The coating layer which lies on the iron oxide core is a cluster of cerium oxide nanoparticles connected together by a polymer matrix based on an acrylic monomer. This type of coating layer is not very stable, notably in water since the acrylic polymer will be solubilized and the cerium oxide nanoparticles will detach from the surface of the core, then allowing the water free access to the iron oxide.

There is therefore a real need to develop metal oxide particles which have a good stability over time, and very particularly a good water resistance, while preserving good optical properties in terms of absorption and/or scattering of light, more particularly of ultraviolet radiation; and also to develop a process capable of preparing such particles.

These objectives are achieved with the present invention, one subject of which is notably a metal oxide particle, in particular of M₁-M₂ oxide type of core/shell structure, comprising a core 1 and one or more upper coating layers 2 covering said core 1, of which:

(i) the core 1 is constituted of oxide of at least one metal M₁, preferably in the crystalline state;

(ii) said upper coating layer(s) 2 cover at least 90% of the surface of the core 1, preferably cover the whole of the surface of the core 1, and comprise one or more inorganic compounds containing one or more elements M₂ and one or more oxygen atoms; and

(iii) said element(s) M₂ are different from the metal(s) M₁ and are chosen from scandium, yttrium, lanthanum, cerium, praseodymium, promethium, samarium, europium, gadolinium, terbium, dysprosium, holmium, erbium, thulium, ytterbium and lutetium, and mixtures thereof; and

it being understood that:
when the core 1 is constituted of titanium oxide and when said upper coating layer(s) 2 are constituted of cerium oxide, then said upper coating layer(s) 2 represent an amount of greater than 1% by weight relative to the total weight of the particle; and
the particle is different from a particle comprising a core 1 constituted of iron oxide Fe₃O₄ and an upper coating layer 2 comprising cerium oxide CeO₂.

It has been observed that the coated metal oxide particles according to the invention only deteriorate very little over time in the presence of water, even when they are formulated in an aqueous composition.

It has also been observed that the metal oxide particles according to the invention have good optical properties in terms of light absorption and/or light scattering. More particularly, they have a high UV absorption and a low visible scattering or a high visible scattering, then allowing uses such as sun protection and/or modification of the visual appearance, while benefiting from resistance in the presence of water.

Moreover, the compositions comprising coated metal oxide particles according to the invention have shown a good screening power, notably with respect to long and short UV-A radiation.

Furthermore, the compositions comprising the coated metal oxide particles of the invention have an especially high transparency, which may prove advantageous when the composition is applied then left to dry on the coating, and in particular on the skin.

Moreover, since the coated metal oxide particles according to the invention do not require a hydrophobic coating, it is possible to use them over a broad formulation spectrum (for example, in entirely aqueous formulations and/or surfactant-free formulations). When the formulations thus obtained end up in water (washbasin drainage, lake or sea), the risk of inappropriate deposit (on the edges of the washbasin, on the walls of the pipes or on rocks) is furthermore reduced.

Another subject of the invention relates to a process for preparing such metal oxide particles, in particular of M₁-M₂ oxide type of core/shell structure, comprising at least the following steps:
a. preparing a composition (A) by adding one or more metal M₁ precursors to a combustible solvent or to a mixture of combustible solvents; then
b. in a flame spray pyrolysis device, forming a flame by injecting the composition (A) and an oxygen-containing gas until aggregates of metal M₁ oxide are obtained; and
c. injecting into the flame a composition (B) comprising one or more element M₂ precursors until a coating layer containing one or more elements M₂ and one or more oxygen atoms is obtained on the surface of said metal M₁ oxide aggregates; said element(s) M₂ being chosen from scandium, yttrium, lanthanum, cerium, praseodymium, promethium, samarium, europium, gadolinium, terbium, dysprosium, holmium, erbium, thulium, ytterbium and lutetium, and mixtures thereof, preferably from cerium, yttrium, lanthanum, and mixtures thereof.

It has been observed that the process according to the invention makes it possible to obtain metal oxide particles coated with a layer of inorganic material based on the element M₂, which are particularly stable over time and have a good water resistance.

Furthermore, unlike conventional coating processes, the process according to the invention has the advantage, despite the presence of the coating, of retaining good intrinsic properties of the centre. Indeed, owing to the specific nature of the coating layer, it is possible, for a given particle weight, to reduce the proportion of metal oxide, without however reducing and/or negatively affecting the properties of said metal oxide.

Thus, the process of the invention makes it possible to produce stable metal oxide particles, while avoiding the inconveniences owing to the increase in the amount of particles which would be conventionally necessary in order to maintain the good optical properties of the metal oxide.

### BRIEF DESCRIPTION OF THE FIGURE

The attached drawing is schematic. The drawing is not necessarily to scale; it is above all intended to illustrate the principles of the invention.

FIG. 1 represents a cross-sectional view of a metal oxide particle according to one embodiment of the invention.

Other subjects, features, aspects and advantages of the invention will emerge even more clearly on reading the description and the example that follows.

In the present description, and unless otherwise indicated:
the expression “at least one” is equivalent to the expression “one or more” and can be replaced therewith;
the expression “between” is equivalent to the expression “extending from” and can be replaced therewith, and implies that the limits are included;
the expression “keratin materials” denotes in particular the skin and also human keratin fibres such as the hair;
the core (1) is also referred to as the “centre”;
the upper coating layers (2) are also referred to as “outer layers”, “shell” or “coating”;
an “alkyl” is understood to mean an “alkyl radical”, i.e. a C₁ to C₁₀, particularly C₁ to C₈, more particularly C₁ to C₆, and preferentially C₁ to C₄, linear or branched hydrocarbon-based radical, such as methyl, ethyl, propyl, isopropyl, n-butyl, isobutyl or tert-butyl;
an “aryl” radical is understood to mean a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl, preferably a phenyl;
an “arylate” radical is understood to mean an aryl group which comprises one or more —C(O)O⁻ carboxylate groups, such as naphthalate or naphthenate;
“complexed metal” is understood to mean that the metal atom forms a “metal complex” or “coordination compounds” in which the metal ion, corresponding to the central atom, i.e. M₁, is chemically bonded to one or more electron donors (ligands);
a “ligand” is understood to mean a coordinating organic chemical group or compound, i.e. which comprises at least one carbon atom and which is capable of coordinating with the metal M₁, and which, once coordinated or complexed, results in metal compounds corresponding to principles of a coordination sphere with a predetermined number of electrons (internal complexes or chelates)—see Ullmann's Encyclopedia of Industrial Chemistry, “Metal complex dyes”, 2005, p. 1-42. More particularly, the ligand(s) are organic groups which comprise at least one group that is electron-donating via an inductive and/or mesomeric effect, more particularly bearing at least one amino, phosphino, hydroxy or thiol electron-donating group, or the ligand is a persistent carbene, particularly of “Arduengo” type (imidazol-2-ylidenes) or comprises at least one carbonyl group. As ligand, mention may more particularly be made of: i) those which contain at least one phosphorus atom —P<i.e. phosphine such as triphenyl phosphines; ii) bidendate ligands of formula R—C(X)—CR′R″—C(X)—R′″ with R and R″″, which are identical or different, representing a linear or branched (C₁-C₆)alkyl group, and R′ and R″, which are identical or different, representing a hydrogen atom or a linear or branched (C₁-C₆)alkyl group, preferentially R′ and R″ represent a hydrogen atom, X represents an oxygen or sulfur atom, or an N(R) group with R representing a hydrogen atom or a linear or branched (C₁-C₆)alkyl group, such as acetylacetone or β-diketones; iii) (poly)hydroxy carboxylic acid ligands of formula [HO—C(O)]n-A-C(O)—OH and the deprotonated forms thereof with A representing a monovalent group when n has the value zero or a polyvalent group when n is greater than or equal to 1, which is saturated or unsaturated, cyclic or non-cyclic and aromatic or non-aromatic based on a hydrocarbon comprising from 1 to 20 carbon atoms which is optionally interrupted by one or more heteroatoms and/or is optionally substituted, notably with one or more hydroxyl groups; preferably, A represents a monovalent (C₁-C₆)alkyl group or a polyvalent (C₁-C₆)alkylene group optionally substituted with one or more hydroxyl groups; and n representing an integer between 0 and 10 inclusive; preferably, n is between 0 and 5, for instance among 0, 1 or 2; such as lactic, glycolic, tartaric, citric and maleic acids, and arylates such as naphthanates; and iv) C₂ to C₁₀ polyol ligands, comprising from 2 to 5 hydroxyl groups, notably ethylene glycol, glycerol, more particularly still the ligand(s) bear a carboxy, carboxylate or amino group, particularly the ligand is chosen from acetate, (C₁-C₆)alkoxylate, (di)(C₁-C₆)alkylamino, and arylate, such as naphthalate or naphthenate, groups;

The term “fuel” is understood to mean a liquid compound which, with dioxygen and energy, is burnt in a chemical reaction generating heat: combustion. In particular the liquid fuels are chosen from protic solvents, in particular alcohols such as methanol, ethanol, isopropanol, n-butanol; aprotic solvents in particular chosen from esters such as methyl esters and those derived from acetate, such as 2-ethylhexyl acetate, acids such as 2-ethylhexanoic acid (EHA), acyclic ethers such as ethyl ether, methyl tert-butyl ether (MTBE), methyl tert-amyl ether (TAME), methyl tert-hexyl ether (THEME), ethyl tert-butyl ether (ETBE), ethyl tert-amyl ether (TAEE), diisopropyl ether (DIPE), cyclic ethers such as tetrahydrofuran (THF), aromatic hydrocarbons or arenes such as xylene, non-aromatic hydrocarbons; and mixtures thereof. The fuels may optionally be chosen from liquefied hydrocarbons such as acetylene, methane, propane or butane; and mixtures thereof.

The Metal Oxide Particles

The metal oxide particle, in particular of M₁-M₂ oxide type of core/shell structure, according to the invention comprises a core 1 constituted of oxide of at least one metal M₁, preferably in the crystalline state.

Preferably, the metal M₁ is chosen from elements from column 2 of the Periodic Table of the Elements, titanium, zinc, copper, scandium, yttrium, lanthanum, cerium, praseodymium, promethium, samarium, europium, gadolinium, terbium, dysprosium, holmium, erbium, thulium, ytterbium and lutetium; more preferentially from magnesium, calcium, titanium, zinc, copper, cerium and yttrium.

According to the invention, the metal(s) M₁ are different from the element(s) M₂.

The crystalline state of the core 1 and also its composition may be determined, for example, by a conventional X-ray diffraction method.

Advantageously, the core 1 of the particle according to the invention is constituted of one or more aggregates of crystalline primary particles of an oxide of at least one metal M₁. In other words, the core 1 is constituted of several microcrystals of an oxide of at least one metal M₁.

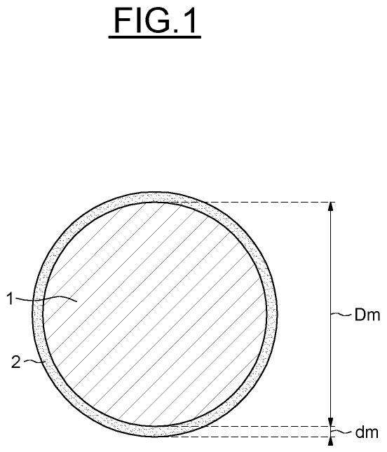

The metal oxide particle according to FIG. 1 comprises a core 1 of average diameter Dₘ, constituted of an oxide of at least one metal M₁ in the crystalline state and comprising one or more aggregates of primary particles of an oxide of at least one metal M₁.

The metal oxide particle according to FIG. 1 also comprises an upper coating layer 2 completely covering the surface of the core 1 and having an average thickness dₘ.

The number-average diameter Dₘ of the core 1 may, for example, be determined by transmission electron microscopy (abbreviated to TEM). Preferably, the number-average diameter Dₘ of the core 1 of the particle according to the invention is within the range extending from 3 to 1000 nm; more preferentially from 6 to 50 nm, and more preferentially still between 10 and 30 nm.

The metal oxide particle according to the invention comprises one or more upper coating layers 2 covering at least 90% of the surface of the core 1.

The degree of coverage of the core by the upper coating layer(s) may for example be determined by means of a visual analysis of TEM-BF or STEM-HAADF type, coupled to a STEM-EDX analysis.

Each of the analyses is carried out on a statistical number of particles, in particular on at least 20 particles. The particles are deposited on a metal grid made of a metal different from any metal that forms part of the particles, whether in the core or in the upper coating layer(s). For example, the grid is made of copper (except in the case where it is desired to use copper in the manufacture of the particles).

Visual analysis of the TEM-BF and STEM-HAADF images makes it possible, based on the contrast, to deduce whether or not the coating completely surrounds the core of the particle. It is possible, by analysing each of the 20 (or more) images, to deduce a degree of coverage of the core, then, by taking the average, to determine an average degree of coverage.

The STEM-EDX analysis makes it possible to verify that the coating does indeed contain predominantly or exclusively the element M₂. For this, it is necessary to make measurements (on at least 20 particles), on the edges of the particles. These measurements then reveal the element M₂.

The STEM-EDX analysis also makes it possible to verify that the core does indeed contain the metal M₁. For this, it is necessary to make measurements (on at least 20 particles), at the centres of the particles. These measurements then reveal the metal M₁ and the element M₂.

Preferably, the upper coating layer(s) 2 completely cover the surface of the core 1.

The upper coating layer(s) 2 comprise one or more inorganic compounds containing one or more elements M₂ and one or more oxygen atoms.

Said element(s) M₂ are different from the metal(s) M₁.

Said element(s) M₂ belong to the group of rare-earth elements in the +III oxidation state, and are chosen from scandium, yttrium, lanthanum, cerium, praseodymium, promethium, samarium, europium, gadolinium, terbium, dysprosium, holmium, erbium, thulium, ytterbium and lutetium, and mixtures thereof.

Preferably, the element(s) M₂ are chosen from cerium, yttrium, lanthanum, and mixtures thereof.

According to a first specific embodiment of the invention, the element M₂ is preferably cerium.

According to another specific embodiment of the invention, the element M₂ is preferably yttrium.

According to yet another specific embodiment of the invention, the element M₂ is lanthanum.

According to yet another specific embodiment of the invention, the metal oxide particles comprise a core 1 constituted of yttrium oxide and an upper coating layer 2 comprising cerium oxide.

The number-average thickness dₘ of the upper coating layer(s) may also be determined by transmission electron microscopy.

Preferably, the number-average thickness dₘ is within the range extending from 1 to 30 nm; more preferentially from 1 to 15 nm, and more preferentially still from 1 to 6 nm.

Advantageously, the upper coating layer(s) 2 are amorphous.

Preferably, the upper coating layer(s) 2 are constituted of one or more oxides of at least one element M₂; said oxide(s) of at least one element M₂ being different from said oxide of at least one metal M₁.

More preferentially, the upper coating layer(s) 2 are constituted of cerium oxide CeO₂, yttrium oxide Y₂O₃, and/or lanthanum oxide La₂O₃, and mixtures of these oxides.

Very particularly preferably, the particle according to the invention comprises an upper coating layer 2 constituted of an oxide of an element M₂ chosen from cerium oxide CeO₂, yttrium oxide Y₂O₃, and/or lanthanum oxide La₂O₃.

Advantageously, the metal oxide particle according to the invention comprises metal M₁ and element M₂ in a specific (M₁/M₂)_{particle} molar atomic ratio for the particle according to the invention.

This ratio corresponds to the amount in moles of metal M₁ atoms present in the particle according to the invention on the one hand, to the amount in moles of element M₂ present in the particle according to the invention on the other hand.

This ratio can be determined by spectrometry according to one of the following two methods. According to a first method, powder is spread out and an X-ray fluorimetry study is carried out with an X-ray spectrometer to deduce therefrom the metal ratio. According to another method, the particles of the invention are dissolved beforehand in an acid. Then an elemental analysis is carried out on the material obtained by ICP-MS (inductively coupled plasma mass spectrometry) to deduce therefrom the metal ratio.

Preferably, the (M₁/M₂)_{particle} molar atomic ratio is greater than or equal to 0.25; more preferentially within the range extending from 0.25 to 99; more preferentially still within the range extending from 1 to 80; and better still within the range extending from 3 to 20.

Preferably, the sum of the content of metal M₁ oxide and the content of element M₂ oxide is at least equal to 99% by weight, relative to the total weight of the core 1 and of the upper coating layer(s) 2.

The number-average diameter of the particle according to the invention may also be determined by transmission electron microscopy. Preferably, the number-average diameter of the particle according to the invention is within the range extending from 3 to 5000 nm; more preferentially from 4 to 3000 nm; and more preferentially still from 5 to 1000 nm.

Preferably, the BET specific surface area of the particle according to the invention is between 1 m²/g and 350 m²/g; more preferentially between 1 m²/g and 200 m²/g; and even more preferentially between 30 and 100 m²/g.

According to a specific embodiment of the invention, the metal oxide particle according to the invention may optionally further comprise an additional coating layer covering the upper coating layer(s) 2 and comprising at least one hydrophobic organic compound.

The hydrophobic organic compound(s) included in the additional coating layer are more preferentially chosen from silicones, in particular silicones comprising at least one fatty chain; carbon-based derivatives comprising at least 6 carbon atoms, in particular fatty acid esters; and mixtures thereof.

The additional coating layer may be produced via a liquid method or via a solid method. Via a liquid method, the hydroxyl functions are reacted with reactive functions of the compound which will form the coating (typically silanol functions of a silicone or the acid functions of carbon-based fatty substance). Via a solid method, the particles are brought into contact with a liquid or pasty compound comprising the hydrophobic substance.

Preferably, the metal oxide particle according to the invention is obtained by the preparation process of the invention as described below.

The Process for Preparing the Coated Metal Oxide Particles

Another subject of the invention relates to the process for preparing the metal oxide particles, in particular of M₁-M₂ oxide type of core/shell structure, comprising at least one step a. of preparing a composition (A), then a step b. of forming the flame, and a step c. of injecting a composition (B).

Step a. of the process according to the invention consists of preparing a composition (A) by adding one or more metal M₁ precursors to a combustible solvent or to a mixture of combustible solvents.

Preferably, the metal M₁ is chosen from elements from column 2 of the Periodic Table of the Elements, titanium, zinc, copper, scandium, yttrium, lanthanum, cerium, praseodymium, promethium, samarium, europium, gadolinium, terbium, dysprosium, holmium, erbium, thulium, ytterbium and lutetium; more preferentially from magnesium, calcium, titanium, zinc, copper, cerium and yttrium.

According to the invention, the metal(s) M₁ are different from the element(s) M₂.

The metal M₁ precursors and the combustible solvents that can be used according to the invention may be chosen from the metal M₁ precursors and the combustible solvents conventionally used in flame spray pyrolysis.

Preferably, the metal M₁ precursor included in the composition (A) comprises one or more metal M₁ atoms optionally complexed with one or more ligands containing at least one carbon atom.

More preferentially, said ligand(s) are chosen from acetate, (C₁-C₆)alkoxylate, (C₂-C₁₀)alkylcarboxylate, (di)(C₁-C₆)alkylamino, and arylate, such as naphthalate or naphthenate, groups.

Preferably, the combustible solvent(s) are chosen from protic combustible solvents, aprotic combustible solvents, and mixtures thereof; more preferentially from alcohols, esters, acids, acyclic ethers, cyclic ethers, aromatic hydrocarbon or arenes, non-aromatic hydrocarbons, and mixtures thereof; and better still from 2-ethylhexyl acetate, 2-ethylhexanoic acid (EHA), ethyl ether, methyl tert-butyl ether (MTBE), methyl tert-amyl ether (TAME), methyl tert-hexyl ether (THEME), ethyl tert-butyl ether (ETBE), ethyl tert-amyl ether (TAEE), diisopropyl ether (DIPE), tetrahydrofuran (THF), xylene, and mixtures thereof.

Very particularly preferably, the combustible solvent(s) are chosen from aprotic combustible solvents comprising at least three carbon atoms and mixtures thereof; and better still from xylene, tetrahydrofuran, 2-ethylhexyl acetate, 2-ethylhexanoic acid (EHA), and mixtures thereof.

Advantageously, the content of metal M₁ precursor in composition (A) is between 1% and 60% by weight and preferably between 15% and 30% by weight relative to the total weight of composition (A).

The preparation process according to the invention further comprises a step b. of injecting composition (A) and an oxygen-containing gas into a flame spray pyrolysis (FSP) device to form a flame.

During this step b., composition (A) and the oxygen-containing gas are advantageously injected into the flame spray pyrolysis device, by two injections that are separate from one another. In other words, composition (A) and the oxygen-containing gas are injected separately, i.e. composition (A) and the oxygen-containing gas are not injected by means of a single nozzle.

More particularly, composition (A) is transported by one tube, whereas the oxygen-containing gas (also referred to as “dispersion Oxygen”) is transported by another tube. The inlets of the two tubes are arranged so that the oxygen-containing gas produces a negative pressure and, via a Venturi effect, causes the composition (A) to be sucked up and converted into droplets.

Step b. may optionally further comprise an additional injection of a “premix” mixture comprising oxygen and one or more combustible gases. This “premix” mixture (also referred to as “supporting flame oxygen”) enables the production of a support flame intended to ignite and maintain the flame resulting from composition A and the oxygen-containing gas (i.e. “dispersion Oxygen”).

Preferably, during step b., composition (A), the oxygen-containing gas, and optionally the “premix” mixture when it is present, are injected into a reaction tube (also referred to as an “enclosing tube”). Preferably, this reaction tube is made of metal or of quartz. Advantageously, the reaction tube has a height of greater than or equal to 30 cm, preferably greater than or equal to 40 cm, and more preferentially greater than or equal to 50 cm. Preferentially, the length of said reaction tube is between 30 cm and 300 cm, particularly between 40 cm and 200 cm, and more particularly between 45 cm and 100 cm, for instance 50 cm.

The weight ratio of the mass of solvent(s) present in composition (A) on the one hand, to the mass of oxygen-containing gas on the other hand, is defined as follows: Firstly, the amount of oxygen-containing gas (also referred to as oxidizer compound) is calculated in order for the assembly formed by composition (A), i.e. the combustible solvent(s) and the metal M₁ precursors(s) on the one hand, and the oxygen-containing gas on the other hand, to be able to react together in a combustion reaction in a stoichiometric ratio (therefore without an excess or deficit of oxidizer compound). Starting from this calculated amount of oxygen-containing gas (also referred to as “calculated oxidizer”), a new calculation is performed to deduce therefrom the amount of oxygen-containing gas to be injected (also referred to as “oxidizer to be injected”), according to the formula: Oxidizer to be injected=Calculated oxidizer/φ with φ preferably between 0.3 and 0.9, and more preferentially between 0.4 and 0.65.

This method is notably defined by Turns, S. R. in An Introduction to Combustion: Concepts and Applications, 3rd ed.; McGraw-Hill: New York, 2012.

The preparation process according to the invention further comprises a step c. comprising the injection, into the flame formed during step b., of a composition (B) comprising one or more element M₂ precursors.

The injection of composition (A) and the injection of composition (B) are preferably simultaneous. In other words, the process of the invention is continuous and the flame formed in step b. is maintained.

Preferably, the flame formed during step b. is at a temperature above or equal to 2 000° C., in at least one part of the flame.

At the site of the injection of the composition (B) into the flame formed in step b. and maintained in step c., i.e. during step c., the temperature is preferably between 200° C. and 800° C., and more preferentially between 400° C. and 500° C.

Advantageously, during step c., composition (B) is injected via a spraying ring, placed above said reaction tube as described above, where in particular the injection of composition (A) takes place. More preferentially, an additional tube is placed in the continuity of said reaction tube and of said spraying ring, the additional tube then being placed above the spraying ring, and the spraying ring being itself placed above said reaction tube.

According to this preference, this additional tube is made of metal or of quartz. Advantageously, this additional tube has the same diameter as said reaction tube and has a height of greater than or equal to 30 cm, preferably greater than or equal to 40 cm, and more preferentially greater than or equal to 50 cm. Preferentially, the length of said additional tube is between 30 cm and 300 cm, particularly between 40 cm and 200 cm, and more particularly between 45 cm and 100 cm, for instance 50 cm.

As indicated above, said element(s) M₂ belong to the group of rare-earth elements in the +III oxidation state, and are chosen from scandium, yttrium, lanthanum, cerium, praseodymium, promethium, samarium, europium, gadolinium, terbium, dysprosium, holmium, erbium, thulium, ytterbium and lutetium, and mixtures thereof.

Preferably, the element(s) M₂ are chosen from cerium, yttrium, lanthanum, and mixtures thereof.

According to a first specific embodiment of the invention, the element M₂ is preferably cerium.

According to another specific embodiment of the invention, the element M₂ is preferably yttrium.

According to yet another specific embodiment of the invention, the element M₂ is lanthanum.

Preferably, the element M₂ precursor(s) comprise one or more atoms of elements M₂ optionally complexed with one or more ligands.

Preferably, the ligand(s) are chosen from acetate, nitrate, (C₁-C₆)alkoxylate, (C₂-C₁₀)alkylcarboxylate, (di)(C₁-C₆)alkylamino, and arylate, such as naphthalate or naphthenate, groups.

The element M₂ precursor(s) may be chosen from the halides of an element M₂.

During the process according to the invention, an (M₁/M₂)_{injected} molar atomic ratio can be calculated. This ratio corresponds to the amount in moles of metal M₁ atoms injected during step b. on the one hand, to the amount in moles of element M₂ injected during step c. on the other hand.

Preferably, the (M₁/M₂)_{injected} molar atomic ratio is greater than or equal to 0.25; more preferentially within the range extending from 0.25 to 120; more preferentially still from 0.25 to 99, better within the range extending from 1 to 80; and better still within the range extending from 3 to 20.

Preferably, nitrogen (N₂) is bubbled into composition (B) as described above, prior to its injection during step c. The rate of injection of composition (B) can then be controlled by control of the temperature and by control of the flow rate of the bubbler.

According to one specific embodiment of the invention, composition (B) as described above is, prior to its injection during step c., brought to a temperature within the range extending from 25° C. to 70° C., more preferentially from 30° C. to 60° C.

Preferably, the content of element M₂ precursor(s) in composition (B) injected during step c. of the process according to the invention is between 1% and 60% by weight, more preferentially between 5% and 30% by weight, relative to the total weight of the composition (B).

Advantageously, composition (B) may also comprise one or more solvents. Preferably, the solvent(s) present in composition (B) are chosen from polar protic solvents other than water; and more preferentially from (C₁-C₈)alkanols. More preferentially still, composition (B) comprises ethanol.

According to a preferred embodiment of the invention, the solvent(s) present in composition (B) are chosen from solvents that are combustible at the flame temperature of step c., preferably from solvents that are combustible at a temperature between 200° C. and 800° C.; and more preferentially between 400° C. and 500° C. Better still, the solvent(s) present in composition (B) have a boiling point above or equal to room temperature (25° C.), or even within the range extending from 50° C. to 120° C.

Preferably, the content of solvent(s) present in composition (B) injected during step c. of the process according to the invention, is between 40% and 99% by weight, more preferentially between 50% and 98% by weight, and better still between 70% and 95% by weight, relative to the total weight of the composition (B).

According to a preferred embodiment of the invention, the preparation process may further comprise a step d. of calcining the metal oxide particles obtained after step c.

According to this embodiment, during the calcining step d.:

(i) the calcining lasts preferably between 60 and 400 minutes, more preferentially between 60 and 180 minutes; and/or

(ii) the temperature ranges preferably from 100° C. to 600° C., more preferentially from 300° C. to 600° C.

According to one specific embodiment of the invention, the particles obtained by the preparation process according to the invention are doped. According to this embodiment, composition (A) further comprises one or more precursors of element D, different from the metal M₁ and from the element(s) M₂, with D chosen from fluorine, vanadium, zirconium, hafnium, iron and tungsten.

Another subject of the invention relates to a composition, preferably a cosmetic composition, comprising one or more metal oxide particles as described above, and/or preferably obtained by the process according to the invention.

The composition of the invention may be in various galenical forms. Thus, the composition of the invention may be in the form of a powder (pulverulent) composition or of a liquid composition, or in the form of a milk, a cream, a paste or an aerosol composition.

The compositions according to the invention are in particular cosmetic compositions, i.e. the material(s) of the invention are in a cosmetic medium. The term “cosmetic medium” means a medium that is suitable for application to keratin materials, notably human keratin materials such as the skin, said cosmetic medium being generally constituted of water or of a mixture of water and of one or more organic solvents or of a mixture of organic solvents.

The composition according to the invention is advantageously an aqueous composition.

Preferably, the composition comprises water in a content notably of between 5% and 95% inclusive relative to the total weight of the composition.

The term “organic solvent” means an organic substance that is capable of dissolving another substance without chemically modifying it.

Examples of organic solvents that may be mentioned include lower C₂-C₆ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

Preferably, the organic solvents are present in the composition according to the invention in a content inclusively between 0.1% and 40% by weight approximately relative to the total weight of the composition, and more preferentially between 1% and 30% by weight approximately and even more particularly inclusively between 5% and 25% by weight relative to the total weight of the composition.

The compositions of the invention may contain a fatty phase and may be in the form of direct or inverse emulsions.

The composition according to the invention may be prepared according to the techniques well known to those skilled in the art, in the form of a simple or complex emulsion (oil-in-water, or abbreviated to O/W, water-in-oil or W/O, oil-in-water-in-oil or O/W/O, or water-in-oil-in-water or W/O/W), such as a cream, a milk or a cream gel.

According to one specific embodiment of the invention, the composition according to the invention may also be in the form of an anhydrous composition, for instance in the form of an oil. The term “anhydrous composition” is intended to mean a composition containing less than 2% by weight of water, preferably less than 1% by weight of water, and even more preferentially less than 0.5% by weight of water relative to the total weight of the composition, or even a composition that is free of water. In compositions of this type, the water possibly present is not added during the preparation of the composition, but corresponds to the residual water provided by the mixed ingredients.

The metal oxide particle(s) according to the invention may also be in dry form (powder, flakes, plates), as a dispersion or as a liquid suspension or as an aerosol. The metal oxide particle(s) of the invention may be used as is or mixed with other ingredients.

Preferably, the compositions of the invention contain between 0.1% and 40% by weight of metal oxide particles of the invention, more preferentially between 0.5% and 20% by weight, more preferentially still between 1% and 10% by weight, and better still between 1.5% and 5% by weight, relative to the total weight of the composition.

Another subject of the invention is the composition according to the invention, preferably a cosmetic composition, for use for protecting the skin, preferably human skin, against visible radiation (i.e. wavelengths between 400 nm and 800 nm) and/or ultraviolet radiation (i.e. wavelengths between 100 nm and 400 nm), UV-A radiation (i.e. wavelengths between 320 nm and 400 nm) and/or UV-B radiation (i.e. wavelengths between 280 nm and 320 nm). The compositions according to the invention make it possible to screen out solar radiation efficiently, they are broad-spectrum, in particular for UV-A radiation (including long-wave UV-A radiation), while being particularly stable over time under UV exposure.

The composition according to the present invention may optionally comprise one or more additional UV-screening agents, other than the metal oxide particle according to the invention, chosen from hydrophilic, lipophilic or insoluble organic UV-screening agents and/or one or more mineral pigments. It will preferentially be constituted of at least one hydrophilic, lipophilic or insoluble organic UV-screening agent.

The compositions of the invention may be used in single application or in multiple application. When the compositions of the invention are intended for multiple application, the content of metal oxide particles of the invention is generally lower than in compositions intended for single application.

For the purposes of the present invention, the term “single application” means a single application of the composition, this application possibly being repeated several times per day, each application being separated from the next by one or more hours, or an application once a day, depending on the need.

For the purposes of the present invention, the term “multiple application” means application of the composition repeated several times, in general from 2 to 5 times, each application being separated from the next by a few seconds to a few minutes. Each multiple application may be repeated several times per day, separated from the next by one or more hours, or each day, depending on the need.

Application Process

The metal oxide particle(s) of the invention are an agent for protecting against UVA and UVB radiation. They notably improve the overall screening-out of UV radiation while maintaining a good overall transmission in the visible range and an excellent transparency in the visible range (400-780 nm).

The metal oxide particle(s) of the invention are notably used in the cosmetic compositions, in particular for application to keratin materials, notably human keratin materials such as the skin, at a concentration preferably between 0.1% and 40% by weight relative to the total weight of the composition comprising them; more preferentially between 0.5% and 20% by weight relative to the total weight of the composition comprising them.

The composition may be in any galenical form.

The metal oxide particle(s) of the invention may be applied to the keratin materials either as a single application or as multiple applications. For example, a cosmetic composition comprising the metal oxide particle(s) of the invention may be applied once.

According to another variant, the application process involves several successive applications on the keratin materials of a cosmetic composition comprising one or more metal oxide particles of the invention.

They may also be connected application methods, such as a saturated single application, i.e. the single application of a cosmetic composition with a high concentration of metal oxide particles according to the invention, or else with multiple applications of cosmetic composition (less concentrated) comprising one or more metal oxide particles of the invention. In the case of multiple applications, several successive applications of cosmetic compositions comprising at least one metal oxide particle of the invention may be repeated with or without a delay between the applications.

Another subject of the invention is a process for treating keratin materials, notably human keratin materials such as the skin, by application to said materials of a composition as defined previously, preferably by 1 to 5 successive applications, leaving to dry between the layers, the application(s) being sprayed or otherwise.

According to one embodiment of the invention, the multiple application is performed on the keratin materials with a drying step between the successive applications of the cosmetic compositions comprising the metal oxide particle(s) of the invention. The drying step between the successive applications of the cosmetic compositions comprising at least one metal oxide particle of the invention may be performed in the open air or artificially, for example with a hot air drying system such as a hairdryer.

Another subject of the invention is the use of the metal oxide particles as described above and/or obtained by the preparation process as described above, for formulating cosmetic or pharmaceutical compositions, in particular having an antiperspirant action or pH-regulating action for the skin, or else intended to protect the skin against visible and/or ultraviolet radiation or to modify the appearance of the skin.

Another subject of the invention is the use of one or more metal oxide particles of the invention as defined above, as UV-A and UV-B screening agent for protecting keratin materials, notably the skin.

The examples that follow serve to illustrate the invention without, however, being limiting in nature.

### EXAMPLES

### Example 1

### 1.1 Firstly, a Composition (A) of Zinc Naphthenate (550 mM) in Xylene was Prepared.

Uncoated zinc oxide particles P1 were then prepared using a conventional FSP preparation process Prep 1 with the pre-prepared composition (A) (outside the invention).

Next, zinc oxide particles coated with cerium dioxide P2 were then prepared using the preparation process Prep 2 according to the invention with the same composition (A) and a composition (B) comprising cerium (III) nitrate hexahydrate (500 mM) and ethanol (invention).

The parameters of the Prep 1 process are the following:
ratio (composition (A)/O₂)=5 mL/min of composition (A) and 7 L/min of gas (O₂). To adjust the oxygen flow rate, φ=0.45 is used.

The parameters of the Prep 2 process are the following:
ratio (composition (A)/O₂)=5 mL/min of composition (A) and 7 L/min of gas (O₂). To adjust the oxygen flow rate, φ=0.45 is used.

In this Prep 2 process, a 40 cm high quartz tube is used to inject composition (A). A spraying ring is placed above the quartz tube in order to inject composition (B). The quartz tube and the spraying ring have a diameter of 10 cm.

In addition, nitrogen is first bubbled through the composition (B). When the composition (B) is injected, the stream of nitrogen heated between 30° C. and 40° C. is adjusted in order to enable the evaporation of the cerium (III) nitrate hexahydrate and so that the (Zn/Ce)_{injected} molar atomic ratio=5.7.

1.2 Once the Particles had been Prepared, it was Observed that the Zinc Oxide Particles Obtained were Crystalline.

Furthermore, the particles obtained according to process Prep 2 according to the invention are coated with cerium dioxide and have a (Zn/Ce)_{particle} molar atomic ratio of 5.7.

The BET specific surface area of the particles according to process Prep 2 is 50 m²/g. The particles according to process Prep 2 have a number-average diameter equal to 22 nm.

### 1.3 Evaluation of the Water Resistance:

A first aqueous suspension S1 (at pH=8, by addition of sodium hydroxide) was prepared from particles P1 and water in a content of 1 g of P1/L of water.
In the same way, a second aqueous suspension S2 (at pH=8, by addition of sodium hydroxide) was prepared from particles P2 and water in a content of 1 g of P2/L of water.

Next, each of the suspensions S1 and S2 were placed in an ultrasound bath for 10 min at a power of 20 W.

The content of Zn²⁺ ions present in the suspensions as a function of time, and relative to the amount of zinc introduced, is then measured by means of a conventional anodic stripping voltammetry method for each suspension.

The results have been collated in the table below:

| | Content of Zn²⁺ (% ions released in a litre of water) | | | | |
|---|---|---|---|---|---|
| Suspensions | at t₀ | at t₀ + 1 h | at t₀ + 2 h | at t₀ + 3 h | at t₀ + 4 h |
| S1 (comparative) | 0 | 60 | 97 | 98 | 98 |
| S2 (invention) | 0 | 5 | 19 | 22 | 23 |

t₀ corresponds to the first measurement carried out less than 10 min after the end of the ultrasound bath.

It should be noted that the coated zinc oxide particles P2 obtained according to the preparation process Prep 2 according to the invention have a much better water resistance than the uncoated zinc oxide particles P1 obtained according to the comparative preparation process Prep 1.

Notably, no selective sedimentation (Ce vs Zn) or selective dissolution was observed for the coated zinc oxide particles P2 (invention) in the suspension S2.

### Example 2

### 2.1 Firstly, a Composition (A) of Zinc Naphthenate (550 mM) in Xylene was Prepared.

Zinc oxide particles coated with cerium dioxide P3 were then prepared using the FSP preparation process Prep 3, with the pre-prepared composition (A) (invention).

The parameters of the Prep 3 process are the following:
ratio (composition (A)/O₂)=5 mL/min of composition (A) and 7 L/min of gas (O₂). To adjust the oxygen flow rate, φ=0.45 is used.

In this Prep 3 process, a 40 cm high quartz tube is used to inject composition (A). A spraying ring is placed above the quartz tube in order to inject composition (B). And an additional 30-cm high metal tube is placed above the spraying ring. The quartz tube, the additional metal tube and the spraying ring all have a diameter of 10 cm.

In addition, nitrogen is first bubbled through the composition (B). When the composition (B) is injected, the stream of nitrogen heated between 30° C. and 40° C. is adjusted in order to enable the evaporation of the cerium (III) nitrate hexahydrate and so that the (Zn/Ce)_{injected} molar atomic ratio=5.7.

Secondly, a portion of the particles P3 prepared was drawn off in order to undergo an additional calcining step at 500° C. for one hour, and thus obtain zinc oxide particles coated with cerium dioxide P4 (invention).

2.2 Once the Particles had been Prepared, it was Observed that the Zinc Oxide Particles P3 and P4 Obtained were Crystalline.

Furthermore, the particles P3 and P4 according to the invention are coated with cerium dioxide and have a (Zn/Ce)_{particle} molar atomic ratio of 5.7.

The BET specific surface area of the particles P3 is 44 m²/g.
The BET specific surface area of the particles P4 is 40 m²/g.
The particles P3 have a number-average diameter equal to 23 nm.
The particles P4 have a number-average diameter equal to 26 nm.

### 2.3 Evaluation of the Water Resistance:

A third aqueous suspension S3 (at pH=8, by addition of sodium hydroxide) was prepared from particles P3 and water in a content of 1 g of P3/L of water.

In the same way, a fourth aqueous suspension S4 (at pH=8, by addition of sodium hydroxide) was prepared from particles P4 and water in a content of 1 g of P4/L of water.

Next, each of the suspensions S3 and S4 were placed in an ultrasound bath for 10 min at a power of 20 W.

The content of Zn²⁺ ions present in the suspensions S3 and S4 and in the suspension 51 of Example 1 above, as a function of time, and relative to the amount of zinc introduced, is then measured by means of a conventional anodic stripping voltammetry method for each suspension.

The results have been collated in the table below:

| | Content of Zn²⁺ (% ions released in a litre of water) | | | | |
|---|---|---|---|---|---|
| Suspensions | at t₀ | at t₀ + 1 h | at t₀ + 2 h | at t₀ + 3 h | at t₀ + 4 h |
| S1 (comparative) | 0 | 60 | 97 | 98 | 98 |
| S3 (invention) | 0 | 3 | 18 | 19 | 20 |
| S4 (invention) | 0 | 1 | 15 | 15 | 16 |

t₀ corresponds to the first measurement carried out less than 10 min after the end of the ultrasound bath.

It should be noted that the coated zinc oxide particles P3 and P4 according to the invention have a better water resistance than the comparative uncoated zinc oxide particles P1.

Notably, no selective sedimentation (Ce vs Zn) or selective dissolution was observed for the coated zinc oxide particles P3 and P4 (invention) respectively in the suspensions S3 and S4.

### Example 3

The particles P1, P2, P3 and P4 from the preceding two examples are used.

200 mg of one type of particles P1 to P4 are introduced into 1 L of water to produce the formulations F1 (based on particles P1), F2 (based on particles P2), F3 (based on particles P3) and F4 (based on particles P4). Then a spectrum is produced in the UV and visible zone for these formulas F1 to F4.

The following absorbances are noted.

| | Absorbance | | |
|---|---|---|---|
| Formulas | 365 nm | 300 nm | 280 nm |
| F1 (comparative) | 1.25 | 1.31 | 1.47 |
| F2 (invention) | 0.45 | 0.47 | 0.52 |
| F3 (invention) | 0.55 | 0.57 | 0.64 |
| F4 (invention) | 1.63 | 1.51 | 1.80 |

It is noted that formulas F2 and F3 according to the invention absorb less UV radiation than formula F1 (comparative).

It is observed that formula F4 according to the invention absorbs more UV radiation than formula F1 (comparative).

A RAMAN study of the particles P1 to P4 was carried out. The Raman peak of the ZnO of the particles P4 is much more intense (around 3 times more) than that of the reference ZnO.

The Raman peaks of the ZnO of the particles P2 and P3 are also observed.

Thus, the invention makes it possible to adjust the screening power of a composition while having, in all cases, a good stability in water.

1. Metal oxide particle comprising a core (1) and one or more upper coating layers (2) covering said core (1), characterized in that:
(i) the core (1) is constituted of oxide of at least one metal M₁, preferably in the crystalline state,
(ii) said upper coating layer(s) (2) cover at least 90% of the surface of the core (1), preferably cover the whole of the surface of the core (1), and comprise one or more inorganic compounds containing one or more elements M₂ and one or more oxygen atoms; and
(iii) said element(s) M₂ are different from the metal(s) M₁ and are chosen from scandium, yttrium, lanthanum, cerium, praseodymium, promethium, samarium, europium, gadolinium, terbium, dysprosium, holmium, erbium, thulium, ytterbium and lutetium, and mixtures thereof; and
it being understood that:
when the core (1) is constituted of titanium oxide and when said upper coating layer(s) (2) are constituted of cerium oxide, then said upper coating layer(s) (2) represent an amount of greater than 1% by weight relative to the total weight of the particle; and
the particle is different from a particle comprising a core (1) constituted of iron oxide Fe₃O₄ and an upper coating layer (2) comprising cerium oxide CeO₂.

2. Particle according to claim 1, characterized in that the metal M₁ is chosen from elements from column 2 of the Periodic Table of the Elements, titanium, zinc, copper, scandium, yttrium, lanthanum, cerium, praseodymium, promethium, samarium, europium, gadolinium, terbium, dysprosium, holmium, erbium, thulium, ytterbium and lutetium; preferably from magnesium, calcium, titanium, zinc, copper, cerium and yttrium.

3. Particle according to claim 1, characterized in that the element(s) M₂ are chosen from cerium, yttrium, lanthanum, and mixtures thereof.

4. Particle according to claim 1, characterized in that the upper coating layer(s) (2) are constituted of one or more oxides of at least one element M₂; preferably, the upper coating layer(s) (2) are constituted of cerium oxide CeO₂, yttrium oxide Y₂O₃, and/or lanthanum oxide La₂O₃, and mixtures of these oxides.

5. Particle according to claim 4, characterized in that the sum of the content of metal M₁ oxide and the content of element M₂ oxide is at least equal to 99% by weight, relative to the total weight of the core (1) and of the upper coating layer(s) (2).

6. Particle according to claim 1, characterized in that the number-average diameter Dm of the core (1), determined by transmission electron microscopy (TEM), is within the range extending from 3 to 1 000 nm, preferably from 6 to 50 nm, and more preferentially from 10 to 30 nm.

7. Particle according to claim 1, characterized in that the number-average thickness dₘ of the upper coating layer(s) (2), determined by transmission electron microscopy (TEM), is within the range extending from 1 to 30 nm, preferably from 1 to 15 nm, and more preferentially from 1 to 6 nm.

8. Particle according to claim 1, characterized in that the number-average diameter of the particle, determined by transmission electron microscopy (TEM), is within the range extending from 3 to 5 000 nm, preferably from 4 to 3 000 nm, and more preferentially from 5 to 1 000 nm.

9. Process for preparing metal oxide particles as defined in claim 1, characterized in that it comprises at least the following steps:
a. preparing a composition (A) by adding one or more metal M₁ precursors to a combustible solvent or to a mixture of combustible solvents; then
b. in a flame spray pyrolysis device, forming a flame by injecting the composition (A) and an oxygen-containing gas until aggregates of metal M₁ oxide are obtained; and
c. injecting into the flame a composition (B) comprising one or more element M₂ precursors until a coating layer containing one or more elements M₂ and one or more oxygen atoms is obtained on the surface of said metal M₁ oxide aggregates; said element(s) M₂ being chosen from scandium, yttrium, lanthanum, cerium, praseodymium, promethium, samarium, europium, gadolinium, terbium, dysprosium, holmium, erbium, thulium, ytterbium and lutetium, and mixtures thereof, preferably from cerium, yttrium, lanthanum, and mixtures thereof.

10. Process according to claim 9, characterized in that the metal M1 precursor comprises one or more metal M1 atoms optionally complexed to one or more ligands containing at least one carbon atom; preferably said ligand(s) are chosen from the following groups: acetate, (C1-C6)alkoxylate, (C2-C10)alkylcarboxylate, (di)(C1-C6)alkylamino, and arylate such as naphthalate or naphthenate.

11. Process according to claim 9, characterized in that the combustible solvent(s) are chosen from protic combustible solvents, aprotic combustible solvents, and mixtures thereof; preferably from alcohols, esters, acids, acyclic ethers, cyclic ethers, aromatic hydrocarbons or arenes, non-aromatic hydrocarbons, and mixtures thereof; more preferentially, the combustible solvent(s) are chosen from aprotic combustible solvents comprising at least three carbon atoms and mixtures thereof; and better still from xylene, tetrahydrofuran, 2-ethylhexyl acetate, 2-ethylhexanoic acid (EHA), and mixtures thereof.

12. Process according to claim 9, characterized in that the element M₂ precursor(s) comprise one or more element M₂ atoms optionally complexed to one or more ligands; preferably said ligand(s) are chosen from the following groups: acetate, nitrate, (C₁-C₆)alkoxylate, (C₂-C₁₀)alkylcarboxylate, (di)(C₁-C₆)alkylamino, and arylate such as naphthalate or naphthenate.

13. Process according to claim 9, characterized in that the composition (B) comprises one or more solvents; preferably the solvent(s) are chosen from polar protic solvent(s) other than water; more preferentially from (C₁-C₈)alkanols; and better still the solvent is ethanol.

14. Process according to claim 9, characterized in that it further comprises a step (d) of calcining the metal oxide particles obtained after step c; preferably at a temperature within the range extending from 100° C. to 600° C., more preferentially from 300° C. to 600° C.

15. Particle obtained by the process as defined in claim 9.

16. Composition comprising one or more particles as defined in claim 1.

17. Composition as defined in claim 16, for use for protecting the skin, preferably human skin, against visible and/or UV-A and/or UV-B ultraviolet radiation.

18. Use of the particles as defined in claim 1, for formulating cosmetic or pharmaceutical compositions, in particular having an antiperspirant action or pH-regulating action for the skin, or else intended to protect the skin against visible and/or ultraviolet radiation or to modify the appearance of the skin.
